(19) United States
(12) Patent Application Publication
(10) Pub. No.: US 2022/0002652 A1
(43) Pub. Date: Jan. 6, 2022

(54) BIOREACTOR VESSEL FOR AUTOMATED FERMENTATION SYSTEM

(71) Applicant: Culture Biosciences, Inc., South San Francisco, CA (US)

(72) Inventors: William Graham PATRICK, San Francisco, CA (US); Matthew Adams BALL, San Mateo, CA (US)

(21) Appl. No.: 17/395,238

(22) Filed: Aug. 5, 2021

Related U.S. Application Data
(63) Continuation of application No. PCT/US2020/017533, filed on Feb. 10, 2020.
(60) Provisional application No. 62/804,091, filed on Feb. 11, 2019.

(51) Int. Cl. C12M 1/06; C12M 1/00; C12M 1/34; C12M 1/36

(52) U.S. Cl. CPC C12M 27/06; C12M 29/06; C12M 41/32; C12M 41/26; C12M 23/58; C12M 41/36; C12M 41/06; C12M 41/48; C12M 41/12

(57) ABSTRACT

A bioreactor may comprise a head-plate configured to provide coupling features to attach selected components to the bioreactor. The bioreactor may also comprise impeller and sparger, and the sparger is positioned at a pre-determined distance from the impeller based at least in part on a spatial configuration of the head-plate. The bioreactor further comprises a vessel defining a hollow enclosure for receiving a culture medium, and an opening of the vessel is covered by the head-plate.

### CROSS REFERENCE

This application is a continuation of International Patent Application No. PCT/US2020/017533, filed Feb. 10, 2020, which claims priority to U.S. Provisional Application No. 62/804,091, filed Feb. 11, 2019, all of which are incorporated herein by reference in their entirety.

### BACKGROUND OF THE INVENTION

Biological reactions or processes can be carried out in bioreactors on a laboratory or industrial scale. For instance, bioreactors can be used in fed-batch applications or used in continuous batch applications. A bioreactor is a specially designed vessel which is built to support the growth of high concentrations of microorganisms and cells. For example, a bioreactor is usually designed to provide a suitable environment in which an organism can efficiently produce a target product such as cell biomass, metabolite and bioconversion product. Precise environmental control is of considerable interest in fermentations or biological reactions since inaccuracy and/or oscillations may lower the system efficiency, reduce reproducibility and produce undesirable end products.

Bioreactors are designed to consider various requirements to enhance its productivity for various products or applications. In some situations, bioreactors may be used in high throughput, automated fermentation systems that allow for controlled variations in the fermentation process. In some cases, single use bioreactors are designed to facilitate universal use in development, in manufacturing operations, and in commercialization of biologics. However, such single use bioreactors lack scalability to a small scale development model to permit process development while meeting the need for an automated fermentation system.

### SUMMARY OF THE INVENTION

An improved bioreactor for fermentation is provided. Moreover, the present disclosure provides a single use bioreactor with improved scalability, flexibility to accommodate various applications, products, and/or performance.

In one aspect, the present disclosure provides a bioreactor comprising: a vessel defining a hollow enclosure for receiving a culture medium; and a head-plate configured to couple a plurality of components to the bioreactor, wherein the plurality of components comprise an impeller and a sparger that collectively achieve a mixing effect of the culture medium within the vessel based at least in part on a configuration of the head-plate. In some embodiments, the head-plate is formed using three-dimensional (3D) printing or injection molding.

In some embodiments, the head-plate comprises coupling features to position at least one of the plurality of components inside the hollow enclosure of the vessel. In some cases, the coupling features provide a sterile interface to the plurality of components.

In some embodiments, the plurality of components further comprise one or more sensor probes selected from the group consisting of: temperature sensor, dissolved oxygen (DO) sensor, pH sensor, carbon dioxide (COs) sensor, biomass concentration sensor, UV Vis/Raman sensor, visible light camera, infrared spectrum camera, reference light emitters and humidity sensor. In some embodiments, the plurality of components further comprise one or more gas or fluid channels providing fluidic communication with the culture medium contained in the vessel.

In some embodiments, the plurality of components further comprise a condenser. In some cases, the condenser is held in place by the head-plate and is located in close proximity to a gas outlet line. For example, the location of the condenser relative to the gas outlet line is determined by the configuration of the head-plate. In some cases, the condenser is formed using 3D printing and a dimension of the condenser matches an opening of the head-plate.

In some embodiments, a distal end of the sparger is positioned at a pre-determined distance from the impeller via a fixation feature. In some embodiments, the impeller is 3D printed with customized geometrics. In some cases, the mixing effect comprises a flow pattern determined based at least in part on the customized geometrics of the impeller. In some instances, the flow pattern is determined based on a location of the impeller coupled to the head-plate relative to the center of the head-plate.

In some embodiments, the sparger is 3D printed with customized geometrics of an orifice of the sparger. In some cases, the mixing effect is achieved by controlling a location of the orifice of the sparger relative to the impeller.

In some embodiments, the vessel comprises multiple baffles integrally formed with the vessel. In some cases, an arrangement of the multiple baffles, number of the multiple baffles and geometrics of the multiple baffles are selected based on a user determined mixing requirement.

In some embodiments, the bioreactor is single-use or disposable. In some embodiments, the head-plate is configured to automatically identify at least one of the plurality of components. In some cases, the head-plate comprises a reader configured to read a serial number of the at least one of the plurality of components.

In some embodiments, a plurality of the bioreactors are handled by a robot of an automated fermentation system. In some embodiments, the automated fermentation system comprises a camera configured to capture image data of the culture medium contained in the vessel.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a bioreactor, in accordance with embodiments of the invention.

FIG. 2 shows an example of a head-plate of a bioreactor.

FIG. 3 shows an example of 3D printed impeller of a bioreactor.

FIG. 4 shows an example of a vessel of a bioreactor.

FIG. 5 illustrates a schematic block diagram of an exemplary design system, in accordance with embodiments of the invention.

FIG. 6 shows an example of a 3D printer for manufacturing one or more components of a bioreactor.

FIG. 7 shows an example of an automated fermentation system with a plurality of bioreactors.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The present disclosure provides a single use or disposable bioreactor. The provided bioreactor can conveniently adapt to various fermentation requirements, cell culture requirements, applications or user-defined requirements. Various aspects of the invention described herein may be applied to any of the particular applications set forth below.

The provided bioreactor can be used to carry out fermentation processes. Fermentation processes can be used for many applications. For instance, fermentation can be utilized for production of biomass (e.g., viable cellular material), production of extracellular metabolites (chemical compounds), production of intracellular components (e.g., enzymes and other proteins), or transformation of a substrate (e.g., the substrate itself may be metabolized to form a product). Fermentation processes are useful for biological experiments, drug manufacturing, food industry, biofuels, or many other applications. In some instances, it may be desirable to provide automated fermentation systems and methods that allow for low risk of contamination, high levels of accuracy and repeatability, high throughput, controlled variations, quicker turnaround, and/or require less manpower. Precise environmental control is of considerable interest in fermentations since oscillations may lower the system efficiency, reduce reproducibility, reduce the efficacy of the scale-down model and produce undesirable end products. The bioreactor of the present disclosure allows for precise environmental control tailored for various fermentation requirements.

The present disclosure provides bioreactors that can be used in the growth of cells and microorganisms. Bioreactors are usually designed to provide optimum environments or conditions that may allow the growth of the microorganisms to be supported. For example, some of the actuated parameters can include: the agitation speed, the aeration rate, the heating intensity or cooling rate, the nutrients feeding rate, the feed liquid's temperature, the feed gas's temperature, acid or base control, antifoam control, inducing agent control condensation control, control of antibiotic additions, control of sample extraction rate and volume, extraction control, gas mixing control, pressure control, headspace aeration control, and/or overlay solvent for in-situ extraction. Conventional bioreactors may comprise a rigid tank that can be sealed closed. An impeller installed within the tank to aerate and mix the culture. A sparger is mounted on the bottom of the tank and is used to deliver gas to the culture to control the oxygen content and pH of the culture. However, the conventional bioreactors lack the capability to be conveniently customized to meet different conditions or requirements.

Bioreactors of the present disclosure may provide improved flexibility to accommodate various applications or fermentation requirements. In particular, the bioreactor may provide improved modularity with one or more components of the bioreactors capable of being individually modified or customized to meet the fermentation requirements at a fine-tuned level. Moreover, the disclosure is also directed to a single use bioreactor that is well suited to incubating cell cultures and thereafter being disposed. The single use bioreactor of the present disclosure can be scaled to any suitable size. The single use bioreactor may also be designed to fit in an automated fermentation system.

In some embodiments, one or more components of the bioreactor may be manufactured using three-dimensional (3D) printing techniques. The one or more components may include an impeller, a head-plate, a sparging system or a condenser. This may beneficially allow for customizable design of the sparging system (e.g., location of the sparging system, orifice number, orifice geometry, etc), the impeller (e.g., impeller number, location, type, shape and geometry, etc), head-plate and various other components (e.g., location, type and number of sensors, location, size, type and number of feed inputs fitted into the head-plate) at a fine-tuned level. Moreover, geometry and/or volume of the vessel may be scalable, and the vessel may be provided with features for improving gas transfer such as by providing multiple baffles. One or more baffles may be positioned and designed with respect to the vessel to support a desired profile. Other fluidic profile or features may be adopted to improve gas transfer. For instance, desired fluidic profile may include the control of the formation of vortices, turbulent mixing patterns and the elimination of zones where mixing does not occur to achieve improved gas transfer into the liquid.

The term “three-dimensional printing” (also “3D printing”), as used herein, generally refers to a process or method for generating a 3D part (or object). For example, 3D printing may refer to sequential addition of material layer or joining of material layers or parts of material layers to form a three-dimensional (3D) part or structure, in a controlled manner (e.g., under automated control). In the 3D printing process, the deposited material can be fused, sintered, melted, bound or otherwise connected to form at least a part of the 3D object. Fusing the material may include melting or sintering the material. Binding can comprise chemical bonding. Chemical bonding can comprise covalent bonding. Examples of 3D printing include additive printing (e.g., layer by layer printing, or additive manufacturing). The 3D printed components may go through further processes such as subtractive manufacturing via manual or automated tooling.

3D printing may be performed using various materials. In some examples, a material that may be used in 3D printing includes a polymeric material, elemental metal, metal alloy, a ceramic, an allotrope of elemental carbon, or a combination thereof. The material may comprise an organic material, for example, a polymer or a resin. The material may comprise a solid or a liquid. The solid material may comprise powder material. The powder material may be coated by a coating (e.g., organic coating such as the organic material (e.g., plastic coating)). The material may be in the form of a powder, filament, wire, pellet, or bead.

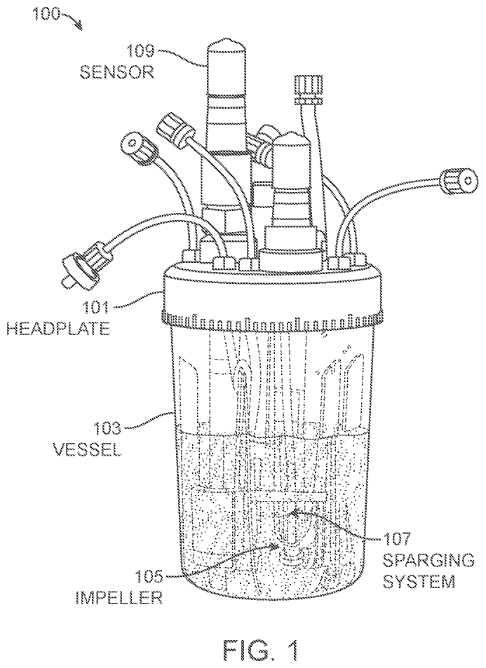

FIG. 1 shows an example of a bioreactor 100, in accordance with embodiments of the invention. The bioreactor may comprise a head-plate 101 with one or more features or ports to fit components (e.g., gas inlets/outlets, feed inputs, sensors, actuators, condenser, etc), a vessel 105, a sparging system 107 and other suitable components. In some embodiments, at least the head-plate, the sparging system and the impeller are 3D printed to advantageously provide flexibility for accommodating different applications or fermentation requirements. For example, a 3D printed head-plate may be configured to provide features to couple selected components to the bioreactor such as specialty sensors that are designed to examine a particular cell type or extracellular product. A 3D printed head-plate may also be configured to allow for additional, individual inputs for substrate, acid, base, antibiotics, nutrients, metals, extracts, antifoam, culture, inoculum and/or inducing agents. In another example, a 3D printed sparger may be positioned at a controlled distance from the impeller according to a user-defined mixing requirement. The 3D printed sparger may be adjusted to control the size of gas bubbles to improve aeration rates or to simulate limited aeration that is found in a production-scale bioreactor. The 3D printed sparger may be designed to deliver multiple independent gas streams with different orifice characteristics. The 3D printed sparger may be designed to deliver gases at different locations throughout the reactor. The 3D printed sparger can be quickly adjusted in response to reactor characterization data, simulation data (e.g. computational fluid dynamic simulations) and demands of the process (e.g. specific oxygen transfer minimum and maximum limits and rates of change, gas mixing concentration parameters, and aeration bubble diameter limits). In a further example, the vessel may define a hollow enclosure for receiving a culture medium, where the vessel may comprise multiple baffles integrally formed with the vessel. The vessel baffles can be adjusted in number, position and profile. They can support a desired fluidic profile including the formation of vortices and turbulent mixing. Details about the head-plate, sparger and the vessel are described later herein.

In some embodiments, the bioreactor 100 may be a single use bioreactor or disposable bioreactor. In some cases, the whole bioreactor is disposable. In some cases, some of the components of the bioreactor are disposable while others are re-usable. Alternatively or in addition to, the bioreactor 100 can be re-usable. For instance, the whole bioreactor or one or more components of the bioreactor can be reused for multiple times.

Head-Plate

In some embodiments, a head-plate 101 of the provided bioreactor may be 3D printed. Alternatively or in additional to, the head-plate may be fabricated using other fabrication methods such as injection molding. The head-plate may cover the vessel and may be removable from the bioreactor or vessel 103. The head-plate allows full access to the vessel. The head-plate may be engaged with the vessel to cover an opening of the vessel. The head-plate may have suitable structures to fit with one or more components of the bioreactor or provide a coupling interface to couple one or more components to the bioreactor. For example, the head-plate may provide openings or one or more ports for coupling probes/sensors 109, such as temperature probes, pH probes, dissolved oxygen (DO) probes, near-infrared spectroscopy (NIR) probes, Fourier transform infrared (FTIR) probes, Raman spectroscopy sensors, biomass sensors, pressure sensors, off-gas sensing probes, couplings for mass spectroscopy connections and/or humidity sensors to the bioreactor. The head-plate may also provide features to fit with components such as condenser system, an impeller, and/or a sparger that can be controlled to achieve a desired fermentation condition. The head-plate may also provide gas or fluid channel providing fluidic communication with the culture medium contained in the vessel and couple various other components to the bioreactor. In some embodiments, the head-plate may provide couplings features for mass spectroscopy connections, visible light cameras, infrared spectrum cameras, reference light emitters and/or humidity sensors. It should be noted that one or more of the components (e.g., temperatures probes, pH probes, dissolved oxygen (DO) probes) may be coupled to the bioreactor through an interface not located at the head-plate. For instance, one or more of the sensor probes may be directly attached to the sidewall of the vessel to be held in place.

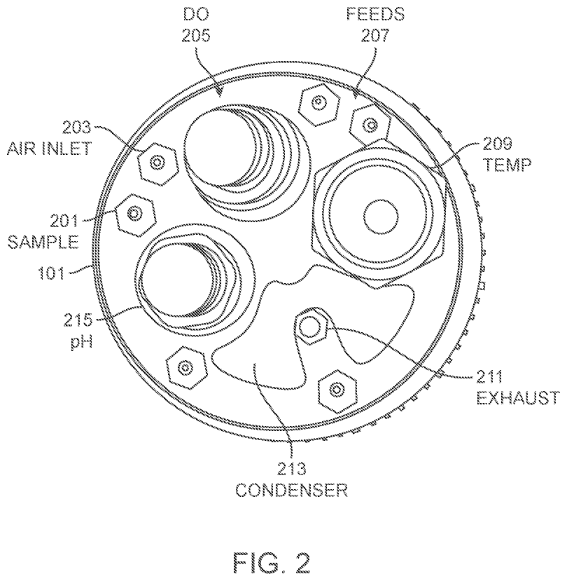

FIG. 2 shows an example of a head-plate 101 of a bioreactor. The head-plate may be 3D printed. The head-plate may be 3D printed to provide means for exit of exhaust gases, ports for addition of liquids or feed inputs, interface for coupling sensors to the bioreactor. Locations and geometries of the various openings, coupling features may be conveniently designed and implemented in aid of 3D printing techniques to accommodate different fermentation requirements or user preferences.

As described above, the head-plate can be formed of any suitable materials. For example, the head-plate may be formed of polymeric materials, vinyl (such as polyvinyl chloride), Nylon (such as vestamid, grilamid), pellethane, polyethylene, polypropylene, polycarbonate, polyester, silicon elastomer, acetate and so forth. Given different use applications such as disposable or re-usable, sterilization methods, fermentation conditions and the like, the materials may be selected such that the materials may be substantively not corrosive, may be capable of tolerating high pressure, may be able to resist pH changes, may be able to tolerate sterilization via the application of steam, irradiation or gas, and/or may be free of toxins or materials that may react to a component or substrate from the fermentation process.

One or more sensors can be coupled to a bioreactor via the head-plate 101. For example, the head-plate may be configured to couple one or more of the following sensors to the bioreactor: temperature sensor 209, dissolved oxygen (DO) sensor 205, pH sensor 215, carbon dioxide (CO2), biomass concentration sensor (e.g., may measure optical density or other characteristics), UV Vis/Raman sensor, scales, mass spectroscopy, camera (e.g., visible light camera, IR spectrum camera), reference light emitters and/or humidity sensors. Sensors may be reusable or may be single-use sensors. The sensors may be positioned inside the vessel or external to the vessel. The sensors may be able to measure a quality of one or more components of the bioreactor or of the fermentation agent within the bioreactor, such as the vessel, sampling location, media culture, or any other component of the bioreactor.

The sensors may be locked into place once the sensors are connected to the bioreactor. In some embodiments, the head-plate may comprise a sterile interface when coupling/attaching or decoupling/detaching the sensors. For example, the head-plate may comprise connectors having a sterile interface in direct contact with the sensors thereby maintaining integrity of the sterile field inside the vessel. In some cases, an inner diameter of the port (of the connector) may be designed to match different diameters of tubes or pipes of the sensors to serve as a sealed barrier.

One or more sensors can be coupled to the bioreactor via the head-plate manually or automatically. In some cases, one or more sensors may be coupled to the headplate in an automated fashion. For instance, the head-plate may comprise a detection feature to automatically identify an identity of the one or more sensor (e.g., Serial Number, sensor ID) and track the port the sensor is attached to. The detection feature can be implemented using any suitable electronics such as sensors. Suitable sensors such as Dallas chip, EEPROM, RFID, barcode and the like may be utilized to track the sensor probe identity, location and/or usage of the sensor probes. In an example, a barcode or RFID (e.g., Serial Number) configured to be read by a reader may be located on the sensor probe and can be read by a reader located on the head-plate.

The head-plate 101 may be conveniently designed and manufactured to adapt to different number of sensors, geometries of sensors, or location of the sensors. Similarly, the head-plate 101 may be conveniently designed and manufactured to fit in or coupled to components such as sample inlet 201, air inlet 203, feeds inlet 207, exhaust line 211 and various others. The head-plate may have various configurations to arrange the one or more sensors in place. Different configurations of the head-plate may determine the location of a component relative to another component or the vessel once the components are coupled to the head-plate. In some cases, spatial configuration of the coupling features (e.g., distance between ports, holes, connectors, locations of the ports relative to each other/vessel center, etc.) in the head-plate may be designed to achieve different configurations of the one or more components spatial arrangements. For instance, a location of a sparging system may be determined by positioning the port corresponding to the sparger at a pre-determined distance from the impeller such that the distal end of the sparger may be preferably positioned below the impeller so as to vent the pumped gas into the area swept by the impeller. The pre-determined distance may be controlled by at least varying the spatial arrangement of the coupling features (e.g., distance between the two ports) corresponding to the sparger and the impeller respectively and by controlling a position of the distal end of the sparging system. For example, a position of the distal tip of the sparger relative to the headplate or the impeller is controlled by attaching the distal portion of the sparger to the distal end of one or more probes via a fixation feature. The fixation feature may be a rigid structure such as a clip to hold the distal end of the sparger in place. In another example, the diameter of the port may be designed to match different diameters of tubes or pipes to meet various gas flow control requirements.

The head-plate 101 embodied herein may provide coupling means to couple the aforementioned components to the bioreactor. The various components such as sensors/probes, gas inputs, feed inputs may be removably assembled to the bioreactor via any suitable mechanical coupling means. For instance, the one or more sensors such as temperature probe 209, the DO probe 205, and the pH probe 215, may be screwed into the threaded feature of the head-plate. Alternatively, the various components may be coupled to the head-plate via other locking features without thread. For instance, the gas inlet or feed tubes may be self-tapping or pressed into the head-plate. In other instances, one or more components may be welded, brazed, or soldered into the head-plate. An adhesive may be used and the adhesive may exhibit bio-inert, non-reactive, high temperature tolerance, resistance to caustic, acidic, basic or corrosive substrates. A manufacturing process such as ultrasonic welding or co-molding may be used to couple components onto the head-plate.

The coupling features may also provide a sterile interface as described above. The coupling features may comprise the connectors as described above. The coupling features may permit the one or more sensors to be connected to the head-plate manually or in an automated fashion. For example, one or more sensors may be assembled to the head-plate with aid of a robotic arm. In another example, an identity of the sensor (e.g., Serial Number) may be identified automatically by a sensor (e.g., RFID transmitter) located at the head-plate as described above. In some cases, the one or more sensors may be assembled to the headplate to form an assembly then the assembly may be coupled to the vessel. Alternatively or in addition to, one or more sensors may be assembled to the head-plate after the head-plate is attached to the vessel.

In some cases, when fermentations operate at high temperatures or contains volatile compounds, a condenser may be required to prevent evaporation loss. A condenser may be provided on a gas outlet line. The condenser may be built into the head-plate itself and may act upon the headspace gas in the bioreactor or in close proximity to the gas outlet line. The condenser may prevent undesired liquid loss from the materials within a vessel. For example, the condenser may prevent undesired water loss from a fermentation broth. In the illustrated example, the head-plate 101 may have an opening or penetrating feature 213 to mate with a condenser 213 so as to allow water flow through the condenser that chills down the head-plate as well as condenses the hot, humid air in the head space.

The condenser may have any suitable shape or geometries. The condenser may be located on the gas exit line (e.g., close to exhaust 211) and configured to reduce the loss of water by evaporation, as shown in FIG. 2. Any number of condensers can be fitted to head-plate. The head-plate may be adjustable to accommodate any number of condensers, location of condensers and size of the condensers. In some cases, multiple condensers may be arranged into optimal locations relative to the vessel or one another for an improved cooling effect.

The condenser may be fitted onto the head-plate using reusable connections. The condenser may be pressed into the head-plate via mating features that match the shape and geometrics of the condenser. In some embodiments, the condenser may be replaceable and/or interchangeable. In some cases, the condenser may be sterilizable. Different shapes, sizes, number of condensers may be employed to provide desired cooling effect. For example, a cross-section of the condenser may match the opening or slot in the head-plate while the thickness of the condenser may be variable to adjust the cooling efficiency.

In some embodiments, the condenser may be 3D printed to advantageously provide varied cooling efficiencies. In some embodiments, the condenser may be formed of metallic material. In some cases, the material for 3D printing the condenser may comprise metal wire that may include, for example, stainless steel, copper, and/or aluminum. Examples of 3D printing methodologies for printing the condenser may comprise extrusion, wire, granular, laminated, or power bed and inkjet head 3D printing. Extrusion 3D printing can comprise robo-casting, fused deposition modeling (FDM) or fused filament fabrication (FFF). In some cases, the 3D printing may be performed without extrusion. For example, beads may be deposited (e.g., in FFF) in a manner that does not involve extrusion. Wire 3D printing can comprise electron beam freeform fabrication (EBF3). Granular 3D printing can comprise direct metal laser sintering (DMLS), electron beam melting (EBM), selective laser melting (SLM), selective heat sintering (SHS), Joule Heating, or selective laser sintering (SLS). Power bed and inkjet head 3D printing can comprise plaster-based 3D printing (PP). In some cases, gas metals arc welding (GMAW), resistive spot welding (RSW), and computer-aided manufacturing (CAM) technologies may be employed for printing a 3D metallic structure. For example, inert gas shielding and a fine metal wire electrode may be utilized as both an electrode and source of metal feedstock (similarly to GMAW), an electric current that heats and melts the feed metal and base metal due to contact resistance (similarly to RSW), and can control the motion of the metal wire electrode/feedstock in three dimensions through a computer-controlled interface, allowing for deposition of material in the desired shape (similarly to CAM).

Impeller

An impeller of the provided bioreactor may be customizable to provide various functions and performance, including but not limited to, bulk fluid and gas-phase mixing, air dispersion, oxygen transfer, heat transfer, shear stress control, tip speed control, cavitation control, suspension of solid particles and maintenance of a uniform environment throughout the vessel contents, or enhancement of mass transfer between dispersed phases. Impellers are usually designed to homogenously mix cells, gases, and nutrients throughout the culture vessel. The mixing action evenly distributes oxygen and nutrients to cells for healthy growth, keeps them from settling to the bottom of the vessel, and helps to maintain a uniform culture temperature. A wide range of impeller designs may be selected from to achieve desired mixing which may be imparted as a radial flow, axial flow, or a combination of the two. The impeller of the provided bioreactor can be designed to be any type depending on the product, application or mixing requirement. For instance, the impeller can have flat blade disk turbine, curved blade disk turbine, pitched blade turbine, curved blade turbine, marine propeller, large pitch blade impeller, intermig, gate with turbine, maxblend, helical ribbon, axial flow hydrofoil impeller, Rushton turbine. The impeller of the provided bioreactor can also have various customizable geometrics or designs. For example, geometrics of the pitch, propeller, blades or the like can be customized.

The impeller may be 3D printed which allows for customized design and convenient variations of a design of the impeller. For instance, the orientation of an impeller, positioning of the blades to the shaft, individual design of each blade and the like can be conveniently modified, simulated with computation fluid dynamics (CFD) models and tested with 3D printing techniques. For example, radial flow occurs when fluid is pushed away from the impeller's axis toward the vessel wall. Axial flow occurs when fluid is pushed up or down along the axis or shaft of the impeller. The orientation of an impeller (left-or right-handed) and its agitating direction determine whether the direction of axial flow is up or down. A right-handed impeller option may push fluid in an upward direction toward the top of the vessel if agitation is clockwise (as viewed from the top). A left-handed option paired with a clockwise agitation may push fluid down toward the bottom of the vessel. By 3D printing the impeller with different orientations, different flow pattern can be achieved. In another example, the impeller may be designed and 3D printed such that blades are positioned on the impeller shaft at different directions for testing different flow patterns and agitation results. In a further example, to increase mixing action in some applications, the impeller may be designed and 3D printed such that one impeller blade may be oriented for up flow while the other is positioned for down flow. By 3D printing the impeller, various agitation effects and mixing requirements can be conveniently achieved, and new form of impeller or improved agitation effects may be explored. Impellers can be designed to mimic the fluid flows experienced in larger reactors to create a more realistic model of those scaled-up systems. For example, the impellers may be designed to intentionally create a heterogeneous environment.

Additionally, the location of the impeller relative to the center of the head-plate or bioreactor may also be designed to achieve different flow patterns or mixing effect. The impeller may be mounted off-center, mounted centrally or in any suitable location. For instance, when the impeller is eccentrically positioned, a single impeller mounted off-center may allow a contiguous change in operating volume during a fed-batch process without having to consider the impact of the liquid surface being cut by the un-submerged rotating impeller.

In some cases, depending on the type of impeller, number of impellers, location of the impeller, orientation, turning direction, speed and other factors, one or more parameters of 3D printing may be adjusted to improve the performance of the impeller. For example, the off-center mounted impeller may rely on a vortex of liquid around the impeller zone to create a net axial flow around the liquid bulk. This vortex, however, can create cyclic strains on the impeller shaft, which can lead to material fatigue and failure. Therefore this mode of mixing may be limited to relatively low agitation rates and average energy dissipation rates, which may result in bioreactors that are less well mixed than those stirred bioreactors able to operate at higher agitation rates and P/V. Such load requirement (e.g., stress directions) may be conveniently taken into account in 3D printing processes. During 3D printing, stress analysis may be conducted so as to improve the property or performance of the printed 3D impeller. For example, composite material may be utilized to provide anisotropic stiffness, strength, thermal and electrical properties to produce high performance designs. The strength in the fiber direction of a fiber composite material is much greater (perhaps ten times greater, in some cases) compared to the transverse direction. In this case, if the high stress directions are known from calculations (such as finite element analysis) the fiber direction can be aligned to provide a much stronger structure compared with many other materials. During 3D printing, the tools paths may be generated according to the complex geometric, structural, mechanical, thermal and/or electrical conductivity properties of the material and the performance of the design. The performance of the impeller can be improved efficiently tailored to different stress/load conditions or mixing requirements.

Moreover, with 3D printing techniques, an impeller can be easily designed, fabricated, tested and the fermentation result may be used as feedback to further improve the design thereby achieving an optimal fermentation condition. Such method may also allow for exploring new forms of impeller that may not be achieved with conventional design and simulation method.

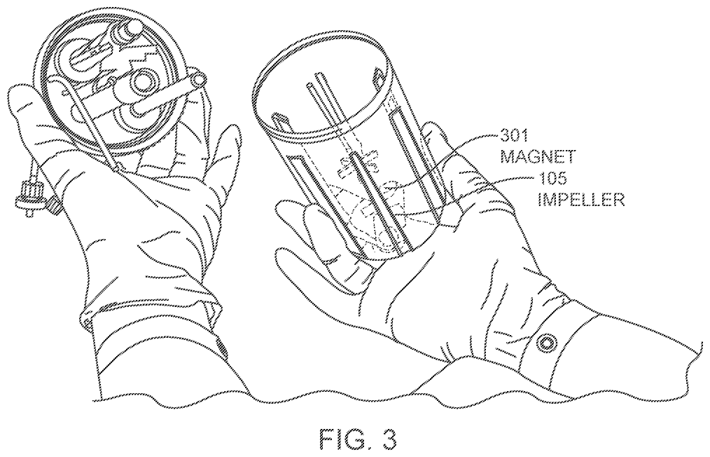

FIG. 3 shows an example of 3D printed impeller 105 of a bioreactor. In some embodiments, the impeller may be driven by a variable-speed DC motor via direct or magnetic coupling. In some cases, the impeller may be driven by a multi-phase AC motor via direct or magnetic coupling. In some cases, the impeller may comprise embedded or built-in magnets 301 to be driven by the motor. The impeller may comprise any number of embedded magnets. For example, one, two, three, four, five, six or more magnets may be embedded in the impeller. The impeller may be 3D printed so that the magnets may be fitted into the impeller in any suitable locations. In some cases, the impeller may have a structure such as a docking feature, a slot, a conduit and the like to hold the magnets in place. The magnets may be coupled to the impeller with or without additional attachment means. The magnets may be attached as part of the impeller's molding process. The impeller may have embedded metals that are later magnetized, after manufacturing.

Sparging System

A sparging system or a sparger is a device for introducing air into the bioreactor. In some cases, gas under pressure is supplied to the sparger (usually either a ring with holes or a tube with single orifice). The provided sparger can be any type such as a porous sparger, an orifice sparger (a perforated pipe), a nozzle sparger (an open or partially closed pipe), and a user-defined or customized sparger.

Referring back to FIG. 1, in some embodiments, the sparging system 107 or the sparger may be 3D printed. The sparger may be formed of substantially rigid materials such that the position of the sparger relative to the impeller can be precisely controlled. For example, the sparger may be formed of polymeric materials, vinyl (such as polyvinyl chloride), Nylon (such as vestamid, grilamid), pellethane, polyethylene, polypropylene, polycarbonate, polyester, silicon elastomer, acetate and so forth. Given different use applications such as disposable or re-usable, sterilization methods, fermentation conditions or other factors, the materials may be selected such that the materials are substantively not corrosive, may be capable of tolerating high pressure, may be able to resist pH changes, may be able to tolerate steam sterilization, may not react or bind with a fermentation product or substrate and/or may be free of toxins.

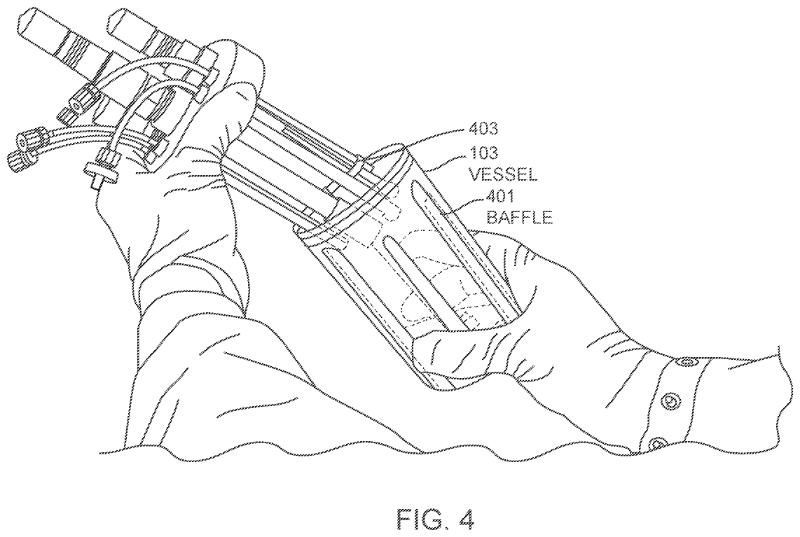

The sparging system and the impeller may operate collectively to achieve a desired fermentation condition or mixing effect. In some cases, the geometrics of the impeller and/or relative position between the sparging system and the impeller may be designed to achieve various mixing effect (e.g., flow pattern). For instance, an impeller may be designed to have a configuration which has a high shear region around the sparger, and/or the sparger may be designed such that the location of the orifice (where gas bubbles exit) relative to the impeller is precisely controlled. 3D printing the sparger may also provide flexibility for adjusting one or more properties of the sparger so as to affect the performance or to provide different fermentation conditions. For instance, the sparger may be varied to have sparge holes being positioned at a pre-determined distance from the inner edges of the impeller blades, variable orifice diameters to choose from to determine the maximum gas flow, or the sparger inlet pipe being placed so as to allow free draining back into the vessel. For example, a position of the distal tip of the sparger relative to the headplate or the impeller is controlled by attaching the distal portion of the sparger to the distal end of one or more probes via a fixation feature. The fixation feature may be a rigid structure to hold the distal end of the sparger in place. In some cases, the fixation feature may be a clip 403 as shown in FIG. 4 to position the distal end of the sparger relative to the probes, head-plate or impeller.

Vessel

The head-plate of the bioreactor is designed to provide full access to the vessel, by its removal from the top of the bioreactor. FIG. 4 shows an example of a vessel 103, in accordance with embodiments of the invention. The vessel 103 may have a volume between about 5 mL and about 50,000 L. Non-limiting examples include a volume of greater than, less than, or equal to about 5 mL, 10 mL, 20 mL, 50 mL, 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. The volume may fall within a range between any two of these values.

The vessel may be formed from a material that is substantively not corrosive, may be capable of tolerating high pressure, may be able to resist pH changes, may be able to tolerate steam sterilization, and/or may be free of toxins. The vessel can be multi-use, single-use, disposable, or non-disposable and can be formed of any suitable material including metal alloys such as stainless steel (e.g., 316L or any other suitable stainless steel) and Inconel, plastics, and/or glass.

In some cases, the vessel may be constructed from plastic-based materials. In some cases, disposable vessels may be constructed of rigid polycarbonate-based plastics for a relatively small volume and may be constructed from flexible low-density polyethylene-based plastics for a relatively greater volume. These materials of construction may have different extractable/leachable profiles of components; these different profiles which may affect the growth, metabolism, excretion, solubility or synthesis of proteins by the cells in different ways. The hydrophobicity of these materials of construction is also different, as is their ability to adsorb hydrophobic components present within the medium. As such, based on the material of construction used in the bioreactor vessel, the feeding and/or the production of actively-growing cells on the material surface are potentially different.

The vessel may be composed of an optically transmissive material, such as one or more of a translucent material, a transparent material, a semi-transparent material, or a semi-translucent material such that content in the vessel can be visible. This may beneficially allow for capturing image data of the vessel and the content contained therein so that real-time information about the fermentation status can be obtained in a non-contact manner.

In some cases, the vessel may be fabricated by injection molding to form a user-defined profile or geometries. For instance, geometries of the vessel, external and inner surface of the vessel can be customized. In some embodiments, the vessel may include one or more baffles 401. In some cases, the vessel may comprise multiple baffles configured to extend adjacent to the side wall of the vessel in a longitudinal direction. The baffle may have a shape that extends radially inward from the side wall an amount sufficient to affect fluid flow in the hollow enclosure during mixing of a culture media by the impeller. The baffles may provide additional mounting points for sensors, probes and other actuators such as heating and cooling elements. The baffles may be actuators themselves, capable of adjusting their profile, length and number in response to dynamically changing mixing and aeration rate control profiles within the vessel.

In some cases, the shape of the baffle, number of baffles, geometrics of a baffle, and arrangement of multiple baffles relative to one another or relative to the vessel may be designed to achieve an optimal performance. The bioreactor, in some embodiments, may include six baffles that are spaced around a circumference of the hollow enclosure of the vessel. The multiple baffles may be spaced apart evenly. Alternatively, the multiple baffles may be spaced apart at variable distances. In some cases, a baffle of the presenting disclosure may have a curved profile. In some cases, the number of baffles or geometrics of baffles may vary according to vessel scale or other fermentation requirements such as mixing time or cellular residence time requirements. In some cases, the shape of the baffle, number of baffles, geometrics and arrangement of multiple baffles may be tested and optimized for a small-scale system and then be applied to a large system. Data from a larger system may inform the design of the smaller scale baffles.

The baffle may be integral with the vessel. In some cases, the plurality of baffles may be formed with the vessel. Alternatively, the baffles can be separate from the vessel. In some cases, the baffles may be removable from the vessel. In some cases, the baffles may be modular that can be assembled, configured, installed or disassembled to the vessel with or without a tool. The baffles may be affixed to the head-plate or they may be affixed to a probe.

In some cases, one or more sensors may be assembled to the headplate to form an assembly then the assembly may be coupled to the vessel. Alternatively or in addition to, one or more sensors may be assembled to the headplate after the headplate is attached to the vessel.

Fabrication of a Bioreactor

As described above, one or more components of the bioreactor can be customized for a user-preferred performance, varied for exploring new forms of bioreactor, or other purposes. In some embodiments, a system for fabricating the bioreactor may be provided. The system may allow a user to input requirements of the bioreactor system at various levels. For instance, a user may input requirements at the system level such as cell lines or microorganism, cost, throughput, volume of a bioreactor, single-use or re-usability of a bioreactor, single-use or re-usability of selected components of a bioreactor, sterilization method, number of impellers, types of sensors, number of feed inlets, type, size, and position of a selected impeller, desired impeller power input, or other requirements. In some cases, a user-provided input to select which components (e.g., impeller, sparging system, vessel, head-plate, condenser) to be customized, modified or designed. In some cases, a user may input requirements at a part or component level. A user may provide desired parameters such as mixing rates, cellular residence times, impeller tip speed, power number, temperature profiles or CFD data. For instance, a user may access a design environment such as a web-based CAD platform or the like that users can employ to modify designs and submit designs of components to printing resources.

In some embodiments, the design and manufacturing process may be a semi-automated process. A user may be guided to customize a bioreactor or components of a bioreactor by the system. For instance, upon receiving a user input indicating a cell line or throughput, the system may automatically retrieve an initial or reference design from a library. The user may then choose to modify or change the reference design. The reference design may be selected from a database that stores a plurality of reference designs under various categories. For example, an impeller reference design may be selected from a category of impeller, and similarly a head-plate reference design may be selected from a collection of head-plate references. The plurality of designs may be categorized by many other ways such as function. Any suitable classification means may be adopted according to the specific design product. Alternatively, the design process may not start with an initial design selected from a database, in this case, an initial model may be manually created from scratch. For example, a head-plate design model may be generated in any available structural design software program, such as AutoCAD, Autodesk, Solid Works, pro/Engineer or Solid Edge. Optionally, the head-plate design model may be generated in a simple, custom design tool tailored to the 3D-printed bioreactor components design.

In some embodiments, a reference design stored in the database may comprise a parametric CAD (computer aided design) model. The parametric CAD model may include a parametric description of the model. For example, for an impeller design, the parametric description of the impeller may include its structure, blades, shaft, orientation, and the like. In some embodiments, it may include three-dimensional descriptions of each component and how they are attached with each other. In some embodiments, it may also include materials properties such as glass, metal and plastic used in the model.

A user may be permitted to modify or create a design of a selected component for 3D printing. A user may input one or more design parameters, create or modify a model via any suitable design environment as described above. In some cases, a user may be permitted to select one or more types of sensors for a bioreactor. For instance, upon receiving a user input indicating a selection of one or more types of sensors, a head-plate reference design may be automatically generated with configurations or features to fit the one or more selected sensors.

In some cases, the numerical control programming codes for the 3D part model or the job may be generated and the numerical control programming code file may be transmitted to a 3D printer for execution.

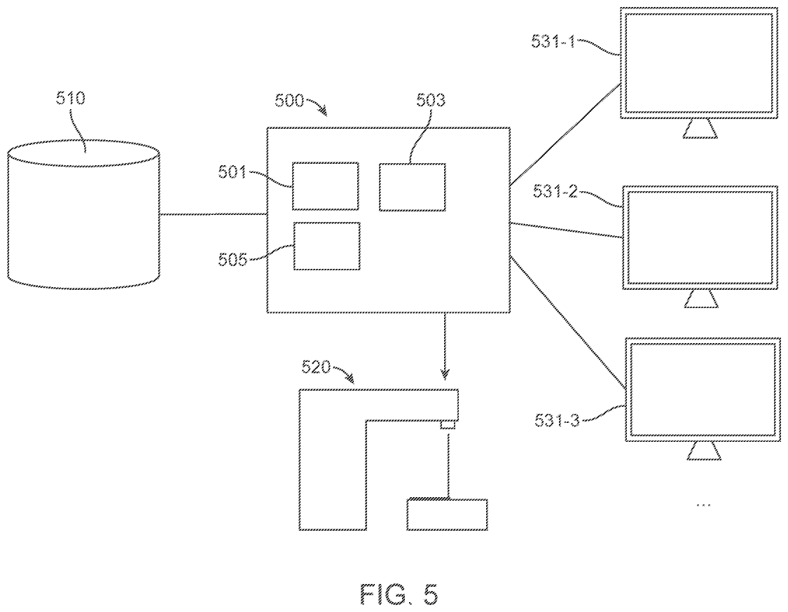

FIG. 5 illustrates a schematic block diagram of an exemplary design system 500, in accordance with embodiments of the invention. The design system may be configured to perform design as described elsewhere herein. The design system may comprise a device including one or more processors 501, a memory 503, a graphical user interface 505, and user interactive device. The memory may comprise non-transitory computer readable media comprising code, logic, or instructions for performing one or more steps, such as the design steps or computations. The memory may comprise one or more databases as described for storing design models, parameters, codes, history performance of parts, history fermentation results as described above. The processor may be configured to perform the steps in accordance with the non-transitory computer readable media. The graphical user interface and user interactive device may allow a user input preferences and requirements to the design as described above. The device may be a desktop computer, cell, smartphone, tablet, laptop, server, or any other type of computational device.

In certain embodiments, the device may be a cloud-based processing cluster implemented on a server configured to operate as a front-end device, where the front-end device is configured to provide a graphical user interface to a user. A server may include known computing components, such as one or more processors, one or more memory devices storing software instructions executed by the processor(s), and data. A server can have one or more processors and at least one memory for storing program instructions. The processor(s) can be a single or multiple microprocessors, field programmable gate arrays (FPGAs), or digital signal processors (DSPs) capable of executing particular sets of instructions. Computer-readable instructions can be stored on a tangible non-transitory computer-readable medium, such as a flexible disk, a hard disk, a CD-ROM (compact disk-read only memory), and MO (magneto-optical), a DVD-ROM (digital versatile disk-read only memory), a DVD RAM (digital versatile disk-random access memory), or a semiconductor memory. Alternatively, the methods disclosed herein can be implemented in hardware components or combinations of hardware and software such as, for example, ASICs, special purpose computers, or general purpose computers.

As mentioned previously, the server may be a server in a data network such as cloud computing network. The server can be computer programmed to transmit data, accept requests, distribute work with other computing devices and/or user interface. In addition, the server may include a web server, an enterprise server, or any other type of computer server.

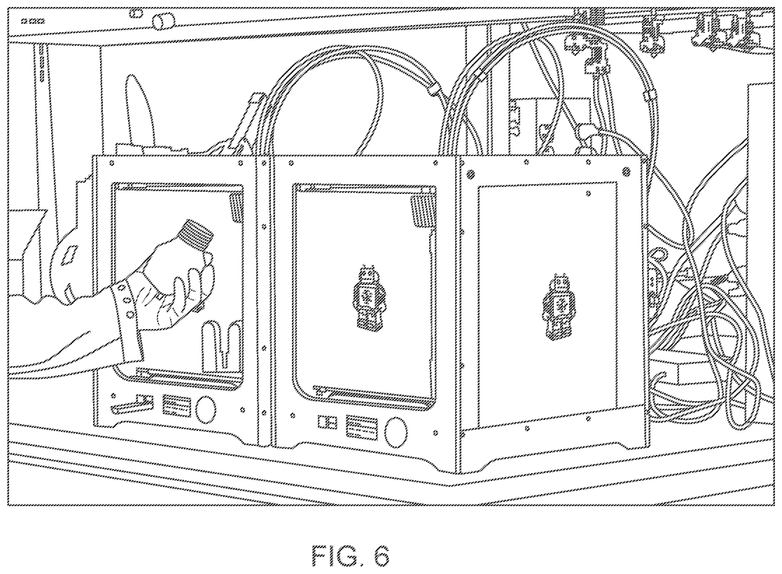

The device may be in communication with a 3D printer 520. FIG. 6 shows an example of a 3D printer. The device may or may not be co-located with the 3D printer. The 3D printer 520 may print the structures according to the design developed in the software program. The 3D printer can be configured to generate an object through additive and/or subtractive manufacturing. The 3D printer can be configured to form a metallic, composite, or polymer object. The 3D printer may be a direct metal laser sintering (DMLS) printer, electron beam melting (EBM) printer, fused deposition modeling (FDM) printer, or a Polyjet printer. The 3D printer may print components such as impeller, head-plate, sparger, condenser or other components made of stainless steel, structural plastics, or any other structural material. As mentioned previously, the system may further comprise a database 510. The database may be accessible to the device and may provide a library storing model copies, history design models, history performance, printing parameters, fermentation experiment results and the like.

The design system may communicate with one or more external devices 531-1, 531-2 and 531-3. The one or more external devices may be a computing device configured to perform design analysis, fermentation experiment analysis, fermentation test result analysis, simulations via CFD or optimizations as described elsewhere herein. The various operations may or may not be operated concurrently on the external devices. The external devices may receive instructions, parameters, design model, etc from the design system and output analysis results or any results according to the instructions to the design system. Communications may occur over a network. The network may be a communication network. The communication network(s) may include local area networks (LAN) or wide area networks (WAN), such as the Internet. The communication network(s) may comprise telecommunication network(s) including transmitters, receivers, and various communication channels (e.g., routers) for routing messages in-between. The communication network(s) may be implemented using any known network protocol, including various wired or wireless protocols, such as Ethernet, Universal Serial Bus (USB), FIREWIRE, Global System for Mobile Communications (GSM), Enhanced Data GSM Environment (EDGE), code division multiple access (CDMA), time division multiple access (TDMA), Bluetooth, Wi-Fi, voice over Internet Protocol (VoIP), Wi-MAX, or any other suitable communication protocols.

Bioreactor for Automated Fermentation System

The provided bioreactor can be utilized in any systems or platforms. In particular, the provided bioreactor can be used in an automated fermentation platform. The automated fermentation system may be of any size. For instance, the automated fermentation system may be the size of a facility, a room, a car, a benchtop, or may be a handheld or portable system. The enclosure may enclose the space of a facility, a room, a car, a benchtop, or may be a handheld or easily transportable item. In some instances, the system may be larger than, approximately the same size as, or smaller than a shipping container. One or more dimensions of the system (e.g., length, width, height, diagonal, diameter) may be less than or equal to 1 cm, 2 cm, 3 cm, 5 cm, 10 cm, 20 cm, 50 cm, 1 m, 1.5 m, 2 m, 3 m, 4 m, 5 m, 7 m, 10 m, 12 m, 15 m, 20 m, 25 m, 30 m, 35 m, 40 m, 50 m, 75 m, or 100 m. One or more dimensions of the system may be greater than any of the values provided, or fall within a range between any two of the values provided. The enclosure may have one or more dimensions less than any of the values provided. One or more dimensions of the enclosure may be greater than any of the values provided or fall within a range between any two of the values provided. In some embodiments, a maximum dimension of the system or enclosure (greatest of length, width, or height) may have a value less than any of the values provided, greater than any of the values provided, or falling within a range between any two of the values provided.

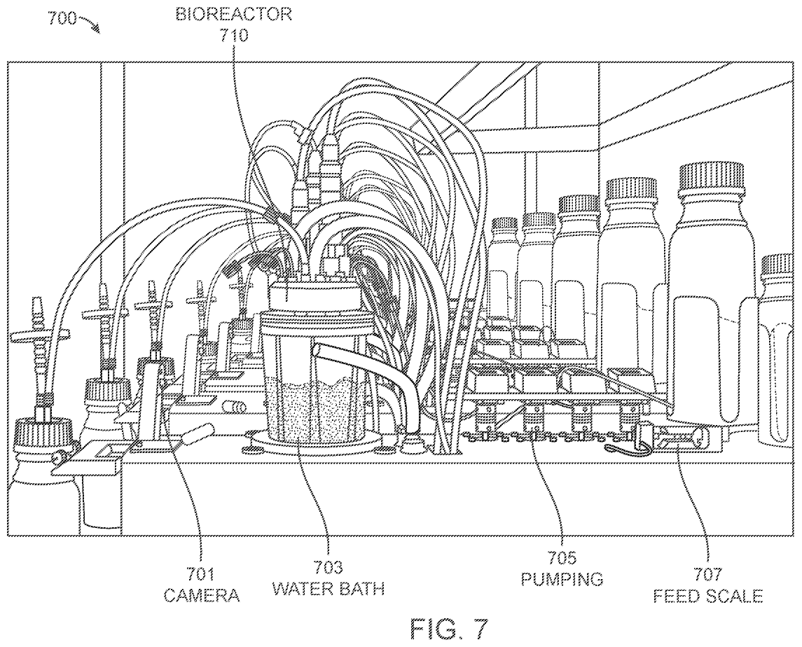

The automated fermentation system may comprise one or more bioreactors provided by the present disclosure. FIG. 7 shows an example of an automated fermentation system 700 with a plurality of bioreactors 710. In some embodiments, a fermentation station may comprise one or more bioreactors. Each bioreactor can correspond to an experiment. Each bioreactor can correspond to an individual fermentation agent consisting of one or more types of cells or microorganisms. Each bioreactor can correspond to an individual fermentation process. Each bioreactor may be independently operating and/or controllable relative to another bioreactor. Any number or arrangement of bioreactors may be provided. The bioreactors used in an automated fermentation system may be individually designed such that the plurality of bioreactors may not be the same. The bioreactors may comprise 3D printed components and a special designed vessel as described above.

One or more modular components may be provided in the automated fermentation system. For instance, components, such as bioreactors and/or other equipment (e.g., analytical equipment, seed train preparation equipment) may be swapped in or out as needed. Modularity may be provided a seed train station, fermentation station, and/or sample handling station. In some embodiments, one or more robotic components may be modular. Such automated fermentation systems include those described in PCT/US2018/061858 entitled “FERMENTATION AUTOMATION WORKCELL”, which is incorporated by reference herein in its entirety.

The provided bioreactors can be conveniently fit in the automated fermentation system and involved in different stages or processes. For instance, one or more bioreactors may be arranged in a bioreactor array on a fermentation station. A bioreactor array may comprise one or more bioreactors. A fermentation station may comprise a plurality of bioreactors. The bioreactors may be arranged in any fashion. A bioreactor array may comprise a single row of bioreactors, multiple rows of bioreactors, a single column of bioreactors, multiple columns of bioreactors, a single stack of bioreactors, or multiple stacks of bioreactors. A bioreactor array may be an m×n array of bioreactors, or an mxnxp array of bioreactors, where m, n, and p are whole numbers of 1 or greater. Optionally, m, n, orp may be greater than or equal to 1, 2, 3, 4, 5, 6, 7, 8, 9, 10, 12, 15, 20, 30, 40, 50, or 100. Optionally, m, n, or p may be less than any of the number provided or fall within a range between any two of the numbers provided.

Any number of bioreactors may be provided within a bioreactor array. For instance, a bioreactor array may comprise 1 or more, 2 or more, 4 or more, 6 or more, eight or more, 10 or more, 12 or more, 18 or more, 24 or more, 36 or more, 48 or more, 60 or more, 96 or more, 128 or more, 256 or more, or any other number of bioreactors. The bioreactor array may comprise less than any of the numbers provided herein, or fall within a range between any two of the numbers provided herein.

Any number of bioreactor arrays may be provided at a fermentation station. For instance, a single bioreactor array may be provided at a fermentation station. Alternatively, a plurality of bioreactor arrays (e.g., two or more, three or more, four or more, five or more) bioreactor arrays may be provided at a fermentation station.

In some embodiments, each bioreactor within a bioreactor array may be capable of operating independently of other bioreactors within the bioreactor array. Each bioreactor array may be capable of operating independently of other bioreactor arrays.

The provided bioreactors can be handled by a robot of the automated fermentation system. A single robot may serve a single bioreactor. Alternatively, a single robot may serve multiple bioreactors. In some instances, a single robot may serve at least 1, 2, 4, 6, 8, 12, 18, 24, 36, 48, 96, 128, 256, or more bioreactors. Alternatively, a single robot may serve fewer bioreactors than any of the values listed herein, or a number of bioreactors falling within a range between any two of the values provided herein. In some embodiments, a single robot may serve a single bioreactor array. Optionally, multiple robots may serve a single bioreactor array. A single robot may serve multiple bioreactor arrays. In some instances, each bioreactor may have one or more dedicated robots. Optionally, multiple robots may be provided that may each serve multiple bioreactor arrays.

A bioreactor utilized in the automated fermentation system may have any dimension. For instance, a bioreactor may have a dimension (e.g., length, width, height, diagonal, or diameter) less than or equal to 1 cm, 3 cm, 5 cm, 10 cm, 15 cm, 20 cm, 25 cm, 30 cm, 35 cm, 40 cm, 45 cm, 50 cm, 60 cm, 70 cm, 80 cm, 90 cm, 1 m, 1.2 m, 1.5 m, or 2 m. A bioreactor may have a maximum dimension less than or equal to any of the values provided herein. A bioreactor may have a dimension greater than any of the values provided herein or falling within a range between any two of the values provided herein. A bioreactor may have a maximum dimension greater than any of the values provided herein, or falling within a range between any two of the values provided herein.

The bioreactor used in the automated fermentation system may be a single-use bioreactor. The bioreactor may be disposable. Alternatively, the bioreactor can be reused. In some embodiments, one or more components of a bioreactor may be single-use or disposable. One or more components of a bioreactor may be reusable. A bioreactor may be removable from fermentation station. A bioreactor may be attached, detached, and/or reattached at the fermentation station. A bioreactor may be exchangeable with another bioreactor. In some embodiments, one or more receiving interfaces may be provided at a fermentation station, which may each be capable of receiving a bioreactor. In some embodiments, each receiving interface may be identical. A bioreactor may fit into any of the receiving interfaces at a fermentation station. Various bioreactors may be received at a receiving interface. In some instances, bioreactors with different settings or different configurations may be received at a receiving interface.

The provided bioreactor can be controlled by the automated fermentation system. The bioreactor may have a control board. The control board may comprise one or more processors that may execute code, logic or instructions to perform one or more steps. The control board may generate instructions that may affect operation of the impeller, the pumps 705, feed inputs, feed scale 707, sparging system, camera 701, sensors, and/or material handling. A control board may optionally control flow of one or more fluids 703. For instance, a control board may control flow of one or more gases into or out of a vessel. A control board may control flow of one or more materials, such as media, to or from the vessel.

The control board may generate instructions that may affect operation of one or more components of the bioreactor. The control board may generate instructions that may affect operation outside the bioreactor. The control board may receive instructions from other sources. For instance, the control board may receive instructions from other bioreactor control boards, from the cloud, from the robots, from any components within the system, or any components outside the system. For example, the instructions may be conveyed to a robot that may interact with the bioreactor. A bioreactor may comprise one or more memory storage units. The one or more memory storage units may comprise non-transitory computer readable media that may comprise code, logic, or instructions for executing one or more steps. The control board may execute one or more experiment protocols. A memory storage unit may store instructions for a particular experiment for the bioreactor.

A temperature control system may be provided for a bioreactor. The temperature control system may comprise a heater and/or cooler that may control the temperature of the bioreactor. The temperature control system may control the temperature of the contents of a vessel. In some embodiments, the temperature control system may be able to provide temperature control to the precision of at least 0.01 degrees C., 0.05 degrees C., 0.1 degrees C., 0.5 degrees C., 1 degree C., 2 degrees C., 3 degrees C., or 5 degrees C. Optionally, a temperature control system may comprise a water bath. A water bath may cool or heat a reactor vessel. In some instances, a temperature control system may comprise thermoelectric heating components. In some instances, Pelletier devices may be used. The temperature control system may comprise a temperature probe held by the head-plate as described elsewhere herein.

A bioreactor may have an on-board camera 701. The on-board camera may be able to visualize the reaction taking place. For instance, the on-board camera may be able to capture images of the contents of the reactor vessel. As described above, the optically transmissive material of the vessel may allow content in the vessel to be visible such that a fermentation status inside the vessel can be analyzed using the image data captured by the on-board camera 701. In some instances, one or more on-board cameras may capture images of the sampling location and/or media containers. An on-board camera may be useful for detecting a stage of an experiment, and/or positioning of any physical components of the bioreactor.

A bioreactor may have a condenser for exhaust gas. In some cases, the condenser may be customized using the aforementioned 3D printing techniques. The condenser may be provided for any type of gas that may be generated within the bioreactor. The condenser may be contained partially or completely within the bioreactor, or externally supported by the bioreactor. The condenser can be the same as the condenser as described above.

In some instances, a bioreactor may comprise a location for ‘run-time’ media additions. In some instances, a media bottle may be provided that may be used for one-time media additions in the middle of an experiment. Any number of containers may be provided that may add materials, such as media, at any point during an experiment, which may occur on the bioreactor. The media additions may be partially or completely within the bioreactor, or supported by the bioreactor. The media additions may be conducted through feed input pipes held by the head-plate of the bioreactor as described elsewhere herein.

A bioreactor may or may not have a local power source. For instance, a bioreactor may have an on-board energy storage system, such as a battery or capacitor. In some instances, a bioreactor need not have an on-board energy storage system and may receive power from an external source. In some instances, a fermentation station receiving interface may provide power to a bioreactor.

A bioreactor may receive inlet gas via an inlet gas interface. Any type of inlet gas may be provided. In some embodiments, the inlet gas may be 02, CO2, air, and/or nitrogen. The flow of gas may be controlled. In some embodiments, the flow of gas may be turned on or off. The rate of gas flow may be controlled (e.g., maintained, increased, or decreased). In some instances, multiple types of gases may be provided. Each type of gas may have its own input interface. Each type of gas may be individually and/or independently controllable. In some instances, a gas inlet may comprise a mixture of gases. The flow of gas may be controlled locally from within the workcell or from outside the workcell. For example, a user at a remote location may control the flow of gas. One or more processors may automatically determine a desired flow of gas, optionally without requiring human intervention. In some embodiments, the inlet gas may enter one or more components of the bioreactor. In one example, the inlet gas may enter a reactor vessel. The inlet gas may aid in a fermentation process. The inlet gas may aid in the calibration of certain probes or actuators (e.g. the gas-sensing probes, product sensing probes, aeration systems, mixing systems or imaging systems). The inlet gas may be used to pressurize a vessel. In some instances, a mass flow controller may control the flow rate of the inlet gas. The mass flow controllers may be provided within each bioreactor.

The source of the inlet gas may be provided within the workcell. Alternatively, the source of the inlet gas may be provided from outside the workcell. The flow of gas may be controlled with aid of one or more pumps, motors, or pressurized reservoirs.

A bioreactor may receive electricity via a power interface. A bioreactor may or may not have a local power source. In some embodiments, a bioreactor may receive power from a power source external to the bioreactor. The power source may be within a workcell. For instance, an energy storage and/or generation system may be provided within a workcell. In some embodiments, a workcell may receive power from outside a workcell. For instance, the workcell may receive power from a utility grid. The electricity provided to the bioreactor may be from outside the workcell, such as a utility grid. In some instances, an entirety of the bioreactor's power may come via the power interface. The bioreactor may be incapable of any operation when detached from the rest of the receiving interface of the workcell. Alternatively, a portion or an entirety of the bioreactor's power may come from a power source on-board the bioreactor. The bioreactor may optionally be capable of some limited operations or regular operation when detached from the rest of the receiving interface of the workcell. In some embodiments, when the bioreactor is attached to the workcell, the electricity may automatically start being provided to the bioreactor. This may optionally start an initialization process. Data may be exchanged between the bioreactor and another portion of the workcell or outside the workcell via the initialization process.

A data communication may occur between a bioreactor and one or more external devices via a data interface. In some embodiments, data communications may be provided to a bioreactor. In some instances, such data communications may include instructions that may pertain to operation of the bioreactor. In some instances, the bioreactor may comprise one or more local controllers that may provide instructions that may affect operation of the bioreactor. The local controllers may receive instructions via data communications that may or may not affect the instructions provided by the local controllers. In some instances, local controllers may not be needed and instructions received by the data communications may directly control operations of the bioreactor.

Data communications may be provided from a bioreactor. In one example, data collected by one or more sensors may be provided from a bioreactor. The data may optionally be provided to one or more external device, such as a computer. Possible communication architectures are provided in greater detail elsewhere herein. In some instances, data communications from the bioreactor may comprise status information about the bioreactor. For example, the data communications may indicate whether the bioreactor is operational, whether the bioreactor is off, whether there is an error state associated with the bioreactor, or any other information. The data communications from the bioreactor may include information about a bioreactor's current stage of an experiment. The data may comprise the bioreactor's estimation of online and offline parameters, as computed from other data and data received by the bioreactor from other sources. The data communications from the bioreactor may include a list or set or prioritized array of errors, warnings and other information that have occurred during an initialization sequence, during experiment execution and/or during shutdown.

Two way communications may be provided via a data interface. In some embodiments, a data interface may detect when a bioreactor is attached to a receiving interface or when a bioreactor is detached from a receiving interface. In some instances, when a bioreactor is attached to a receiving interface an initialization process may occur. For example, the bioreactor may receive power from the power interface and automatically provide information about the bioreactor (e.g., bioreactor identity, bioreactor configurations and modules, bioreactor status, etc.). This may allow for a modular workcell system, where various bioreactors may operate independently of one another and may optionally have different configurations.

A bioreactor may be suspended in water, or another liquid, via a liquid interface 703 for the purposes of temperature control. Any type of liquid may be provided. In some embodiments, the liquid may be water. The water may be a cold water or hot water. In some instances, the liquid may be a coolant. Any coolant known or later developed in the art may be provided, such as ethylene glycol or propylene glycol. The liquid may have a biocide or anti-microbial agent suspended within it such as silver or another compound to prevent cross-contamination. The liquid may be dyed or clear to distinguish it from other liquids such as the fermentation broth. The flow of liquid may be controlled. In some embodiments, the flow of liquid may be turned on or off. The rate of liquid flow may be controlled (e.g., maintained, increased, or decreased). The temperature of the liquid may be controlled (e.g., maintained, increased, or decreased). In some instances, multiple types of liquid may be provided. Each type of liquid may have its own input interface. Each type of liquid may be individually and/or independently controllable. In some instances a liquid input may comprise a mixture of liquids. The flow of liquid may be controlled locally from within the workcell or from outside the workcell. For example, a user at a remote location may control the flow of liquid and/or a desired liquid temperature. One or more processors may automatically determine a desired flow of liquid, optionally without requiring human intervention. In some embodiments, the liquid may enter one or more components of the bioreactor. In one example, the liquid may enter a reactor condenser. In another example, the liquid may enter a water bath that may be used to heat or cool a reactor vessel. The liquid may aid in a fermentation process. The liquid may be provided via a channel, pipe, tubing, or other mechanism.

The source of the liquid may be provided within the workcell. Alternatively, the source of the liquid may be provided from outside the workcell. The flow of liquid may be controlled with aid of one or more pumps. In some instances, one, two or more liquid feed lines may be provided, which may be controlled by one or more pumps, such as peristaltic pumps.

A gas outlet may be provided. Gas from one or more bioreactors may be conveyed outside the various bioreactors. The gas may be conveyed within or outside the workcell. The pressure of the gas may be monitored. In response to certain high pressure measurements, an automated action such as the opening of a release valve, or an alert message, may be sent to an operator. In some embodiments, the gas may come from one or more components of the bioreactor. In one example, the gas may come from a reactor vessel. The gas may be a byproduct of a fermentation process. The gas may be conveyed via a channel, pipe, tubing, or other mechanism. In some instances, fans or pumps may aid in the flow of the outlet gas.

Optionally, a condenser may be provided on a gas outlet line. The condenser may prevent undesired liquid loss from the materials within a reactor vessel. For example, the condenser may prevent undesired water loss from a fermentation broth. The condenser may be 3D printed as described elsewhere herein. The rate of gas flowing through the condenser may be measured. The relative and absolute humidity of the gas flowing through the condenser may be measured. In response to certain measurements, an automated action may take place or an alert message may be sent to an operator.

The outlet gas may optionally be conveyed to an off-gas analysis system. The off-gas analysis system may be provided within a workcell or outside a workcell. The off-gas analysis system may optionally automatically analyze the outlet gas without requiring human intervention. The off-gas analysis system may analyze the outlet gas. In some embodiments, the system may analyze outlet gas mass, composition, quantity, density, temperature, volume, volumetric flow rate, mass flow rate, absolute pressure, partial pressures, absolute humidity, relative humidity or other characteristics. For example, 02 and/or CO2 concentration of live yeast fermentations may be analyzed by the off-gas analysis system for each bioreactor. The off-gas analysis may be performed continuously throughout a fermentation process and recorded by the system.

The systems and methods provided herein may allow for an automated fermentation workcell to perform experiments in a fully automated fashion. This may allow for communication between the various components to perform the fermentation processes, which may include the seed train preparation, the fermentation, sample preparation, and/or sample analysis. The automated fermentation workcell may also allow for testing and optimizing designs of bioreactors by providing real-time sample analysis.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A bioreactor comprising:
a vessel defining a hollow enclosure for receiving a culture medium; and
a head-plate configured to couple a plurality of components to the bioreactor, wherein the plurality of components comprise an impeller and a sparger that collectively achieve a mixing effect of the culture medium within the vessel based at least in part on a configuration of the head-plate.

2. The bioreactor of claim 1, wherein the head-plate is formed using three-dimensional (3D) printing or injection molding.

3. The bioreactor of claim 1, wherein the head-plate comprises coupling features to position at least one of the plurality of components inside the hollow enclosure of the vessel.

4. The bioreactor of claim 3, wherein the coupling features provide a sterile interface to the plurality of components.

5. The bioreactor of claim 1, wherein the plurality of components further comprise one or more sensor probes selected from the group consisting of: temperature sensor, dissolved oxygen (DO) sensor, pH sensor, carbon dioxide (COs) sensor, biomass concentration sensor, UV Vis/Raman sensor, visible light camera, infrared spectrum camera, reference light emitters and humidity sensor.

6. The bioreactor of claim 1, wherein the plurality of components further comprise one or more gas or fluid channels providing fluidic communication with the culture medium contained in the vessel.

7. The bioreactor of claim 1, wherein the plurality of components further comprise a condenser.

8. The bioreactor of claim 7, wherein the condenser is held in place by the head-plate and is located in close proximity to a gas outlet line.

9. The bioreactor of claim 8, wherein the location of the condenser relative to the gas outlet line is determined by the configuration of the head-plate.

10. The bioreactor of claim 7, wherein the condenser is formed using 3D printing and a dimension of the condenser matches an opening of the head-plate.

11. The bioreactor of claim 1, wherein a distal end of the sparger is positioned at a pre-determined distance from the impeller via a fixation feature.

12. The bioreactor of claim 1, wherein the impeller is 3D printed with customized geometrics.

13. The bioreactor of claim 12, wherein the mixing effect comprises a flow pattern determined based at least in part on the customized geometrics of the impeller.

14. The bioreactor of claim 13, wherein the flow pattern is determined based on a location of the impeller coupled to the head-plate relative to the center of the head-plate.

15. The bioreactor of claim 1, wherein the sparger is 3D printed with customized geometrics of an orifice of the sparger.

16. The bioreactor of claim 15, wherein the mixing effect is achieved by controlling a location of the orifice of the sparger relative to the impeller.

17. The bioreactor of claim 1, wherein the vessel comprises multiple baffles integrally formed with the vessel.

18. The bioreactor of claim 17, wherein an arrangement of the multiple baffles, number of the multiple baffles and geometrics of the multiple baffles are selected based on a user determined mixing requirement.

19. The bioreactor of claim 1, wherein the bioreactor is single-use or disposable.

20. The bioreactor of claim 1, wherein the head-plate is configured to automatically identify at least one of the plurality of components.

21. The bioreactor of claim 20, wherein the head-plate comprises a reader configured to read a serial number of the at least one of the plurality of components.

22. The bioreactor of claim 1, wherein a plurality of the bioreactors are handled by a robot of an automated fermentation system.

23. The bioreactor of claim 22, wherein the automated fermentation system comprises a camera configured to capture image data of the culture medium contained in the vessel.
